(19) United States
(12) Patent Application Publication
(10) Pub. No.: US 2020/0279643 A1
(43) Pub. Date: Sep. 3, 2020

(54) DEEP LEARNING FOR MEDICAL DEVICE SELECTION

(71) Applicant: Boston Scientific Scimed Inc., Maple Grove, MN (US)

(72) Inventors: Kyle H. Srivastava, St. Paul, MN (US); Dominic J. Allocco, Edina, MN (US); Paul R. Holleran, Grafton, MA (US)

(21) Appl. No.: 16/514,035

(22) Filed: Jul. 17, 2019

Related U.S. Application Data
(60) Provisional application No. 62/700,043, filed on Jul. 18, 2018.

(51) Int. Cl. G16H 40/40; G16H 30/40; G06T 7/62; G06K 9/62; G06N 20/00

(52) U.S. Cl. CPC G16H 40/40; G16H 30/40; G06N 20/00; G06K 9/6232; G06T 7/62

(57) ABSTRACT

Embodiments of the present disclosure relate to systems and methods for determining the size of a medical device to implant in a patient using deep learning techniques. In at least one embodiment, a method comprises receiving a plurality of images, each of the plurality of images including a representation of a portion of a patient's anatomy in which the medical device is to be implanted. The method further comprises extracting a centerline of the representation of the patient's anatomy from the plurality of images and extracting planes orthogonal to the centerline. In addition, the method comprises identifying, using a segmentation model that segments the extracted planes, an implantation site. And, the method comprises determining, using a medical device size classification model that classifies the implantation site, a size of the medical device to be implanted at the implantation site.

### CROSS REFERENCE TO RELATED APPLICATION

This application claims priority to Provisional Application No. 62/700,043, filed Jul. 18, 2018, which is herein incorporated by reference in its entirety.

### TECHNICAL FIELD

The present disclosure relates to implantable medical devices. More specifically, the present disclosure relates to systems and methods for determining the size of a medical device to implant in a patient using deep learning techniques.

### BACKGROUND

Patients receiving implantable medical devices have widely-varying anatomies. As such, for patients receiving implantable medical devices, physicians will need to select which size medical device to implant into a patient in order to accommodate different patient anatomies. When the implantable medical device is intended to replace a portion of a patient's anatomy, e.g., a heart valve replacement, selecting an appropriately-sized implantable medical device is of particular importance.

### SUMMARY

Embodiments included herein facilitate determining the size of a medical device to implant in a patient using deep learning techniques. Example embodiments are as follows.

In an Example 1, a method for determining a size of a medical device to be implanted in a patient, comprises: receiving a plurality of images, each of the plurality of images including a representation of a portion of a patient's anatomy in which the medical device is to be implanted; extracting a centerline of the representation of the patient's anatomy from the plurality of images; extracting planes orthogonal to the centerline; identifying, using a segmentation model that segments the extracted planes, an implantation site; and determining, using a medical device size classification model that classifies the implantation site, a size of the medical device to be implanted at the implantation site.

In an Example 2, the method of Example 1, the patient's anatomy being an aorta, the area of interest being a representation of an annulus of the aorta, and the size of the medical device being one of three sizes.

In an Example 3, the method of any one of Examples 1-2, further comprising extracting features of the patient's anatomy from the representation of a patient's anatomy using a layer of the medical device size classification model.

In an Example 4, the method of Example 3, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

In an Example 5, the method of any one of Examples 1-4, further comprising training the medical device size classification model using a classification dataset.

In an Example 6, the method of Example 5, further comprising augmenting the classification dataset by randomly flipping, shifting, rotation, zooming, cropping images, and/or adding noise to images included in the classification dataset.

In an Example 7, the method of any one of Examples 1-6, further comprising training the segmentation model using a segmentation dataset by: determining an intensity value for each voxel in each extracted plane of the segmentation dataset; aggregating, for each plane, the intensity values; and identifying local extrema among the aggregated intensity values.

In an Example 8, the method of Example 7, wherein identifying local extrema comprises identifying a local minimum, the local minimum corresponding to an annulus.

In an Example 9, a non-transitory computer readable medium having a computer program stored thereon for determining which size medical device to implant in a patient, the computer program comprising instructions for causing one or more processors to: receive a plurality of images, each of the plurality of images including a representation of a portion of a patient's anatomy in which the medical device is to be implanted; extract a centerline of the representation of the patient's anatomy from the plurality of images; extract planes orthogonal to the centerline; identify, using a segmentation model that segments the extracted planes, and implantation site; and determine, using a medical device size classification model that classifies the implantation site, a size of the medical device to be implanted at the implantation site.

In an Example 10, the non-transitory computer readable medium of Example 9, the patient's anatomy being an aorta, the area of interest being a representation of an annulus of the aorta, and the size of the medical device being one of three sizes.

In an Example 11, the non-transitory computer readable medium of any one of Examples 9-10, the computer program comprising instructions for causing the one or more processors to extract features of the patient's anatomy from the representation of a patient's anatomy using a layer of the medical device size classification model.

In an Example 12, the non-transitory computer readable medium of Example 11, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

In an Example 13, the non-transitory computer readable medium of any one of Examples 9-12, the computer program comprising instructions for causing the one or more processors to train the medical device size classification model using a classification dataset.

In an Example 14, the non-transitory computer readable medium of Example 13, the computer program comprising instructions for causing the one or more processors to train the segmentation model by: determine an intensity value for each voxel in each extracted plane; aggregate, for each plane, the intensity values; and identify local extrema among the aggregated intensity values.

In an Example 15, the non-transitory computer readable medium of Example 14, wherein to identify local extrema, the computer identifies a local minimum, the local minimum corresponding to an annulus.

In an Example 16, a method for determining a size of a medical device to be implanted in a patient, the method comprising: receiving a plurality of images, each of the plurality of images including a representation of a portion of a patient's anatomy in which the medical device is to be implanted; extracting a centerline of the representation of the patient's anatomy from the plurality of images; extracting planes orthogonal to the centerline; identifying, using a segmentation model that segments the extracted planes, an implantation site; and determining, using a medical device size classification model that classifies the implantation site, a size of the medical device to be implanted at the implantation site.

In an Example 17, the method of Example 16, the patient's anatomy being an aorta, the area of interest being a representation of an annulus of the aorta, and the size of the medical device being one of three sizes.

In an Example 18, the method of Example 16, further comprising extracting features of the patient's anatomy from the representation of a patient's anatomy using a layer of the medical device size classification model.

In an Example 19, the method of Example 18, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

In an Example 20, the method of Example 16, further comprising training the medical device size classification model using a classification dataset.

In an Example 21, the method of Example 20, further comprising augmenting the classification dataset by randomly flipping, shifting, rotation, zooming, cropping images, and/or adding noise to images included in the classification dataset.

In an Example 22, the method of Example 16, further comprising training the segmentation model using a segmentation dataset by: determining an intensity value for each voxel in each extracted plane of the segmentation dataset; aggregating, for each plane, the intensity values; and identifying local extrema among the aggregated intensity values.

In an Example 23, the method of Example 22, wherein identifying local extrema comprises identifying a local minimum, the local minimum corresponding to an annulus.

In an Example 24, the method of Example 22, further comprising augmenting the segmentation dataset by randomly flipping, shifting, rotation, zooming, cropping images, and/or adding noise to images included in the segmentation dataset.

In an Example 25, the method of Example 16, further comprising preprocessing the plurality of images, the preprocessing including at least one of: scaling the plurality of images so the plurality of images are uniform and removing at least some portions of the plurality of images that do not include the representation of the patient's anatomy.

In an Example 26, a non-transitory computer readable medium having a computer program stored thereon for determining which size medical device to implant in a patient, the computer program comprising instructions for causing one or more processors to: receive a plurality of images, each of the plurality of images including a representation of a portion of a patient's anatomy in which the medical device is to be implanted; extract a centerline of the representation of the patient's anatomy from the plurality of images; extract planes orthogonal to the centerline; identify, using a segmentation model that segments the extracted planes, and implantation site; and determine, using a medical device size classification model that classifies the implantation site, a size of the medical device to be implanted at the implantation site.

In an Example 27, the non-transitory computer readable medium of Example 26, the patient's anatomy being an aorta, the area of interest being a representation of an annulus of the aorta, and the size of the medical device being one of three sizes.

In an Example 28, the non-transitory computer readable medium of Example 26, the computer program comprising instructions for causing the one or more processors to extract features of the patient's anatomy from the representation of a patient's anatomy using a layer of the medical device size classification model.

In an Example 29, the non-transitory computer readable medium of Example 28, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

In an Example 30, the non-transitory computer readable medium of Example 26, the computer program comprising instructions for causing the one or more processors to train the medical device size classification model using a classification dataset.

In an Example 31, the non-transitory computer readable medium of Example 30, the computer program comprising instructions for causing the one or more processors to augment the classification dataset by randomly flipping, shifting, rotation, zooming, cropping images, and/or adding noise to images included in the classification dataset.

In an Example 32, the non-transitory computer readable medium of Example 26, the computer program comprising instructions for causing the one or more processors to train the segmentation model by: determine an intensity value for each voxel in each extracted plane; aggregate, for each plane, the intensity values; and identify local extrema among the aggregated intensity values.

In an Example 33, the non-transitory computer readable medium of Example 14, wherein to identify local extrema, the computer identifies a local minimum, the local minimum corresponding to an annulus.

In an Example 34, the non-transitory computer readable medium of Example 32, the computer program comprising instructions for causing the one or more processors to augment the segmentation dataset by randomly flipping, shifting, rotation, zooming, cropping images, and/or adding noise to images included in the segmentation dataset.

In an Example 35, the non-transitory computer readable medium of Example 26, the computer program comprising instructions for causing the one or more processors to preprocess the plurality of images, the preprocessing including at least one of: scaling the plurality of images so the plurality of images are uniform and removing at least some portions of the plurality of images that do not include the representation of the patient's anatomy.

While multiple embodiments are disclosed, still other embodiments of the present disclosure will become apparent to those skilled in the art from the following detailed description, which shows and describes illustrative embodiments of the subject matter described herein. Accordingly, the drawings and detailed description are to be regarded as illustrative in nature and not restrictive.

### BRIEF DESCRIPTION OF THE DRAWINGS

FIG. 1 is a classification system for determining a size of a medical device to be implanted in a patient, in accordance with embodiments of the present disclosure.

FIG. 2A is a partial sectional view of an example heart, in accordance with embodiments of the present disclosure.

FIG. 2B is an anterior view of the example heart depicted in FIG. 2A.

FIG. 3 is a partial sectional view of a flattened projection of an example aortic valve, in accordance with embodiments of the present disclosure.

FIG. 4 is a flow diagram of a method for determining a size of a medical device to be implanted in a patient, in accordance with embodiments of the present disclosure.

FIG. 5 is a flow diagram of a method for training a plane segmentation model, in accordance with embodiments of the present disclosure.

FIG. 6A is a computed tomography scan of an exemplary aorta and FIG. 6B is a graph depicting aggregated intensity values for the exemplary aorta, in accordance with embodiments of the present disclosure.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As used herein in association with values (e.g., terms of magnitude, measurement, and/or other degrees of qualitative and/or quantitative observations that are used herein with respect to characteristics (e.g., dimensions, measurements, attributes, components, etc.) and/or ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a value, configuration, orientation, and/or other characteristic that is equal to (or the same as) the stated value, configuration, orientation, and/or other characteristic or equal to (or the same as) a value, configuration, orientation, and/or other characteristic that is reasonably close to the stated value, configuration, orientation, and/or other characteristic, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

### DETAILED DESCRIPTION

As discussed above, physicians will oftentimes need to select which size medical device for implantation for patients receiving implantable medical devices. Selecting the wrong size medical device to implant in a patient can result in adverse effects for the patient. For example, extra surgical time may be needed and/or serious health effects can result from selecting an implantable medical device that does not adequately fit a patient's anatomy. Conventionally, selecting which size medical device to implant depends on pre-screening medical images combined with a physician's intuition. This process, however, has led to inadequately-sized medical devices being implanted in patients. The embodiments disclosed herein seek to solve this problem by recommending which size implantable medical device to select for different patients using deep learning techniques.

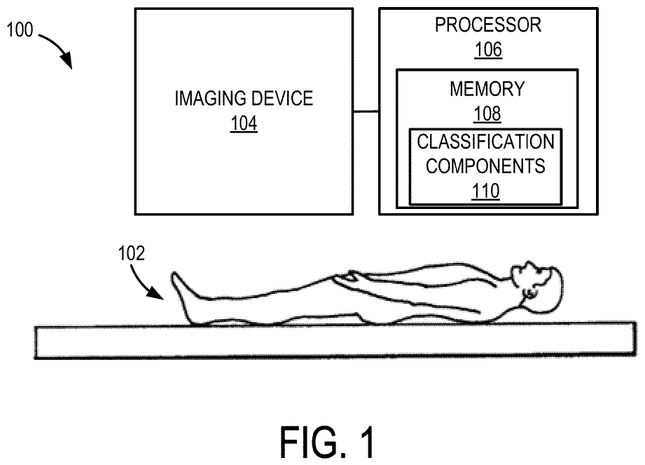

FIG. 1 is a classification system 100 for determining a size of a medical device to be implanted in a patient 102, in accordance with embodiments of the present disclosure. In embodiments, the classification system 100 includes an imaging device 104, a processor 106, memory 108, and one or more classification components 110 stored on memory 108. The imaging device 104 may be arranged external to the patient 102 (as shown) or internal to the patient 102 (not shown). In embodiments, the imaging device 104 may be used to image one or more portions of a patient's 102 anatomy. Example imaging devices 104 include, but are not limited to, ultrasound imaging systems, computed tomography (CT) imaging systems, magnetic resonance imaging systems, positron emission tomography (PET) imaging systems, infrared (IR) imaging systems, and/or the like.

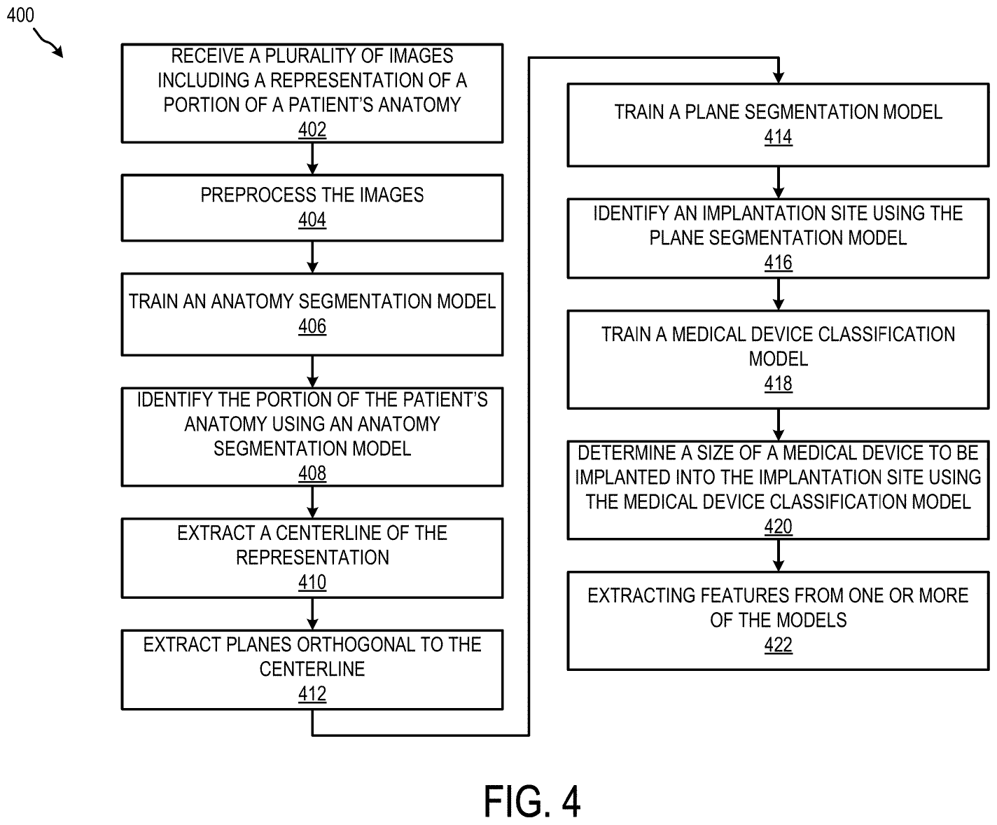

After the imaging device 104 images one or more portions of a patient's 102 anatomy, the processor 106 receives the images and determines a size of a medical device (e.g., a heart valve) to be implanted into the patient 102 using one or more classification components 110 stored on memory 108. The classification components 110 may include a plurality of classification components 110 for performing different classification functions, as described below in relation to FIG. 4.

In embodiments, the processor 106 may include, for example, a processing device, a pulse generator, a controller, and/or the like. The processor 106 may be any arrangement of electronic circuits, electronic components, processors, program components and/or the like configured to store and/or execute programming instructions, to direct the operation of the other functional components of the imaging device 104, the memory 108, and/or the classification components 110. For example, the processor 106 may control the storage of the images obtained by the imaging device 104, control the classification components 110 and/or the like, and may be implemented, for example, in the form of any combination of hardware, software, and/or firmware.

In embodiments, the processor 106 may represent a single processor 106 or multiple processors 106, and the single processor 106 and/or multiple processors 106 may each include one or more processing circuits. In embodiments, the processor 106 may configured to be implanted in the patient and/or may be configured to be outside of the patient. That is, for example, the processor 106 may represent a component of an implantable medical device, an external medical device, and/or may represent at least one processor in an implantable medical device and at least one processor in an external device. The processor 106 may include one or more processing circuits, which may include hardware, firmware, and/or software. In embodiments, different processing circuits of the processor 106 may perform different functions. For example, the processor 106 may include a first processing circuit configured to segment a portion of the patient's 102 anatomy from an image, a second processing circuit configured to identify an implantation site for a medical device, and a third processing circuit configured to determine a size of a medical device to be implanted into the implantation site, which are discussed in further detail below in relation to FIG. 4.

In embodiments, the processor 106 may be a programmable micro-controller or microprocessor, and may include one or more programmable logic devices (PLDs) or application specific integrated circuits (ASICs). In some implementations, the processor 106 may include memory as well. The processor 106 may include digital-to-analog (D/A) converters, analog-to-digital (ND) converters, timers, counters, filters, switches, and/or the like. The processor 106 may execute instructions and perform desired tasks as specified by the instructions.

The processor 106 may also be configured to store information in the memory 108 and/or access information from the memory 108. The memory 108 may include volatile and/or non-volatile memory, and may store instructions (e.g., the classification components 110) that, when executed by the processor 106 cause methods (e.g., algorithms) to be performed, for example, the method 400 depicted in FIG. 4.

The medical device for which the classification components 110 determine a size may be a replacement heart valve. While the discussion herein primarily relates to heart valves, the embodiments apply to other medical devices including, but not limited to, medical devices manufactured (e.g., 3D printed) in different sizes for different sized patients (e.g., stents, other artificial organs, and/or the like.)

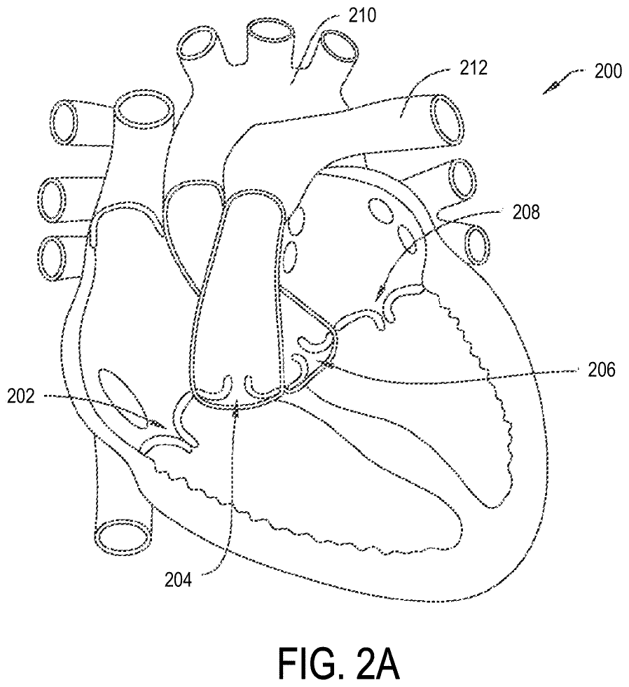

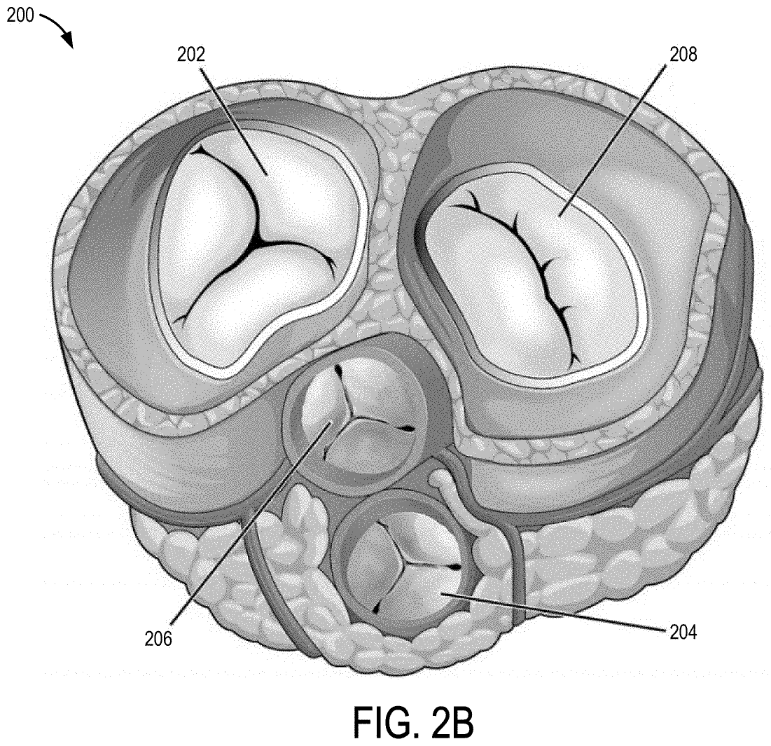

An exemplary portion of a patient's 102 anatomy for which the classification system 100 determines a size of a medical device to be implanted is depicted in FIGS. 2A and 2B. Specifically, FIG. 2A is a partial sectional view of an example patient's heart 200 and FIG. 2B is an anterior view of the example patient's heart 200 depicted in FIG. 2A.

As depicted in FIG. 2A, the patient's heart 200 includes four heart valves: a tricuspid valve 202, a pulmonary valve 204, an aortic valve 206, and a mitral valve 208. The purpose of the heart valves is to allow blood to flow through the heart 200 and from the heart 200 into the major blood vessels connected to the heart 200, such as the aorta 212 and the pulmonary artery 212, for example. In a normally functioning heart valve, blood is permitted to pass or flow downstream through the heart valve (e.g., from an atrium to a ventricle, from a ventricle to an artery, etc.) when the heart valve is open, and when the heart valve is closed, blood is prevented from passing or flowing back upstream through the heart valve (e.g., from a ventricle to an atrium, etc.). Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Furthermore, inadequate sizing of a heart valve can lead to additional surgical time and/or other potentially life-threatening complications. As stated above, the embodiments disclosed herein may facilitate determining a size of a heart valve to be implanted in a patient 102. For the purpose of this disclosure, the discussion below is directed toward a replacement aortic valve 206. This, however, is not intended to be limiting as the skilled person will recognize that the following discussion may also apply to other applications. For example, the following discussion may be applicable to identifying narrowing of an artery and/or implanting a stent therein, replacement of another type of heart valve, and/or implantation of another type of medical device where the implanted site is based on the diameter of the implantation site.

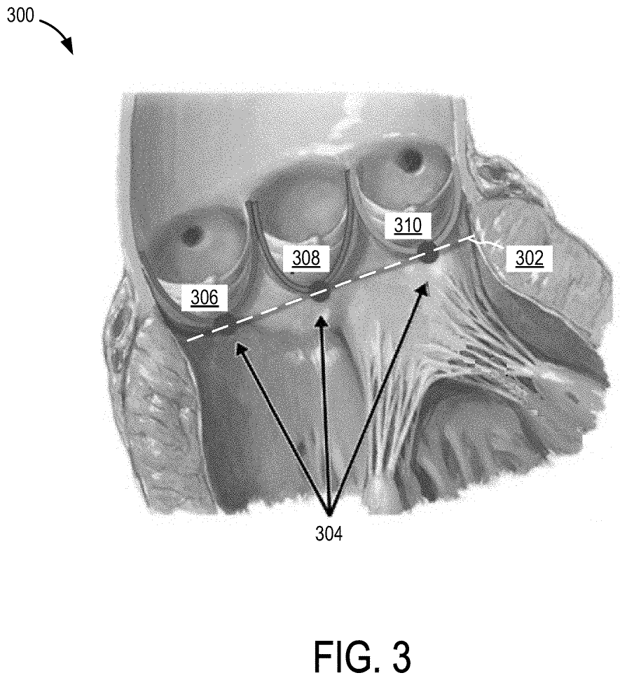

FIG. 3 is a partial sectional view of a flattened projection of an example aortic valve 300, in accordance with embodiments of the present disclosure. To replace the aortic valve 300, the diameter, location (indicated by line 302), amount of calcification and/or other characteristics of the aortic annulus may be determined determined. The aortic valve 206 is cut out along the aortic annulus 302 and replaced with an artificial aortic valve 206. For example, the LOTUS Edge™ valve system manufactured by Boston Scientific may be used to replace the removed aortic valve 206.

As stated above, one or more characteristics (e.g., diameter, location, amount of calcification) of the aortic annulus 302 is determined prior to removal of the aortic valve 206. The aortic annulus 302 is defined as a ring with anchors at the nadirs 304 of the right coronary sinus 306, non-coronary sinus 308, and left coronary sinus 310, which close to form the aortic valve 206. Conventionally, the aortic annulus 302 has been identified by a physician by visually inspecting an image of the aortic valve 206. And, based on the identification of the aortic annulus 302, the size of the replacement aortic valve is approximated by a physician. Various factors, however, make it difficult for a physician to accurately identify the aortic annulus 302. For example, variances among different patients' anatomies, idiosyncratic calcification of aortic valves 300, idiosyncratic trauma to aortic valves 300 and/or the like may make it difficult for a physician to identify the aortic annulus 302 and, therefore, determine the size of the replacement aortic valve.

In embodiments, replacement aortic valves may come in a plurality of different sizes (e.g., 3 different sizes). In the event the size of the aortic annulus 302 is determined incorrectly by the physician and/or an improper size of the replacement aortic valve is selected, additional operating time may be required and/or adverse medical conditions can result. Conventional techniques for determining the size of the aortic annulus 302 (i.e., visual inspection by a physician of one or more images of the patient's 102 aortic valve 206) has led to improperly-sized replacement aortic valves being selected. As such, there is a need in the art to improve upon the embodiments for selecting the size of an aortic replacement valve.

FIG. 4 depicts an exemplary method 400 for facilitating the selection of a size of an aortic replacement valve and/or other medical device. The method 400 may be performed using an imaging system (e.g., the imaging device 104 of FIG. 1). Additionally or alternatively, the method 400 may be implemented on a processor (e.g., the processor 106 of FIG. 1) configured to receive one or more classification components (e.g., the classification components 110 of FIG. 1) stored on memory (e.g., the memory 108).

The method 400 comprises receiving a plurality of images that include a representation of a portion of a patient's anatomy (block 402). In embodiments, an imaging device (e.g., the imaging device 104) may be used to generate the images. For example, one or more of the following imaging systems may be used to generate the images: ultrasound imaging systems, computed tomography (CT) imaging systems, magnetic resonance imaging systems, positron emission tomography (PET) imaging systems, infrared (IR) imaging systems, and/or the like.

In embodiments, the portions of the patient's anatomy depicted in the representations include the portions in which a medical device is to be implanted. For example, the representations may include the patient's aorta. However, the patient's aorta is one example. Other portions of a patient's anatomy can be included in the representations in the event the embodiments disclosed herein are used to determine a size of a medical device to be implanted in other portions of the patient's anatomy.

In embodiments, the representations may be from the same viewpoint or from different viewpoints. In embodiments, the different viewpoints may be combined to yield a three-dimensional representation of a portion of a patient's anatomy. Additionally or alternatively, the different viewpoints may facilitate a more accurate size determination.

In embodiments, the method 400 includes preprocessing the images (block 404). For example, portions of an image not including the representation of the portion of the patient's anatomy may be cropped, filtered, and/or removed from the image. Additionally or alternatively, the images may include portions of the patient's anatomy that are not of interest. The portions of non-interest may also be cropped, filtered, and/or removed. In embodiments, the cropping, filtering, and/or removing of portions of the images may be performed manually and/or automatically using, for example, machine learning. By cropping, filtering, and/or removing portions of the images that are of non-interest, the processing required to train an anatomy segmentation model (block 406) and/or identify the portion of the patient's anatomy using the anatomy segmentation model (block 408) may be reduced.

Additionally or alternatively, the images may be preprocessed by making the images uniform across different scaling factors. Making the images uniform across different scaling factors may facilitate comparing the images with one another and/or comparing the images of a first patient to a second patient.

In embodiments, the preprocessed images may be saved to memory (e.g., the memory 108 depicted in FIG. 1). Additionally or alternatively, the images may be saved to memory prior to being preprocessed.

In embodiments, the method 400 comprises training an anatomy segmentation model (block 406). The anatomy segmentation model may be trained to identify the portion of the patient's anatomy in the images. In embodiments, the anatomy segmentation model may be trained on images included in an anatomy segmentation model dataset. In at least some embodiments, the images included in the anatomy segmentation model dataset may be augmented with random flipping, shifting, rotation, zooming, cropping images, and/or adding noise (e.g., Gaussian noise) to images to improve the likelihood the anatomy segmentation model identifies the portion of the patient's anatomy in the images.

To train the anatomy segmentation model, one or more of the following learning techniques may be used: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style.

In at least some embodiments, one or more models may be used in addition or in alternative to training a model as the anatomy segmentation model. For example, the U-Net or a variation thereof may be used as the anatomy segmentation model. A variation of U-Net is described in, for example, “U-Net: Convolution Networks for Biomedical Image Segmentation,” authored by Olaf Ronneberger, Philipp Fischer, and Thomas Brox, and available at https://pdfs.semanticscholar.org/0704/5f87709d0b7b998794e9fa912c0aba912281.pdf, the entirety of which is hereby incorporated by reference for all purposes. Additionally or alternatively, other types of models for segmenting anatomy in images may be used to train the anatomy segmentation model.

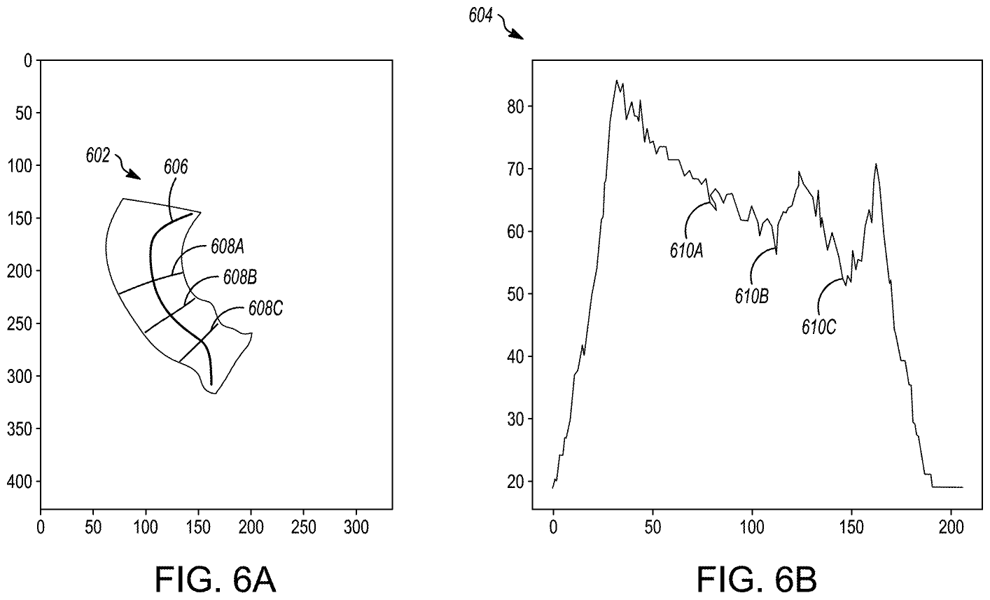

Once an anatomy segmentation model is trained and/or once an anatomy segmentation model is selected, the method 400 may use the anatomy segmentation model to identify a portion of the patient's anatomy in the images (block 408). An exemplary image for which a portion of the patient's anatomy has been identified is depicted in FIG. 6A.

The method 400 may further comprise extracting a centerline of the representation (block 410). The centerline may be extracted using machine learning techniques. For example, a centerline dataset including images where a centerline is identified may be used to train the extraction of a centerline from the identified portion of a patient's anatomy. Additionally or alternatively, the centerline may be extracted using a model trained on one more of the following learning techniques: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style.

In at least some embodiments, the centerline may be straightened to facilitate one or more of the remaining steps of the method 400. For example, straightening the centerline may facilitate extracting planes orthogonal to the centerline, as performed in block 412. In embodiments, the extracted planes may be used to determine the aortic annulus, as described below.

To extract planes orthogonal to the centerline, the ends of the patient's anatomy may be determined from the ends of the centerline. That is, the portions of the patient's anatomy that are orthogonal to the ends of the centerline may be determined to be the ends of the patient's anatomy. Once the ends of the patient's anatomy are determined, a plurality of planes orthogonal to the centerline may be extracted. That is, the patient's anatomy may be divided into a plurality of planes.

In at least some embodiments, the extracted planes may begin at a first end of the patient's anatomy relative to the centerline, extend lengthwise through the centerline, and terminate at a second end of the patient's anatomy that is on the opposite side of the centerline relative to the first end. In embodiments, each of the extracted planes may have the same thickness that extends parallel to the centerline. In at least some embodiments, the thickness and/or the number of extracted planes may vary depending on the size of the representation of the patient's anatomy. For example, if the representation of the patient's anatomy extends parallel to the centerline for X number of pixels, then the patient's anatomy may be divided into Y planes so that each plane is X/Y pixels thick. As an example, a patient's anatomy that extends parallel to the centerline for 150 pixels, the method 400 may include extracted 30 planes orthogonal to the centerline so that each of the extracted planes is 5 pixels thick.

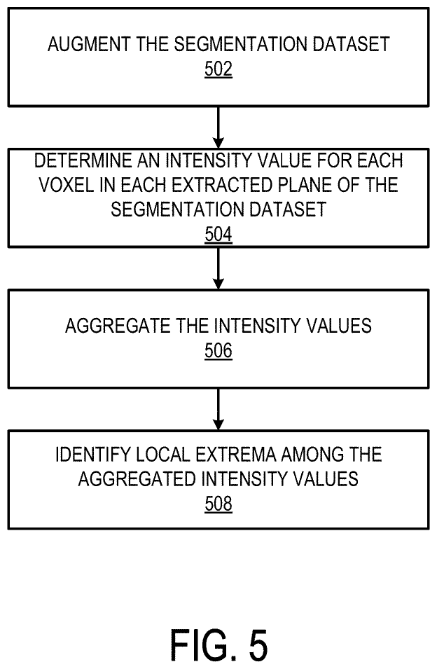

In embodiments, the method 400 may include training a plane segmentation model (block 414). The plane segmentation model may be trained to identify which of the extracted planes includes the aortic annulus. For example, the plane segmentation model may have a binary output that outputs whether the extracted plane includes the aortic annulus or doesn't include the aortic annulus. Additionally or alternatively, the plane segmentation model may output how many planes the extracted plane is away from the annulus. An exemplary method for training the plane segmentation model is described below in relation to FIG. 5.

In embodiments, the plane segmentation model may be trained on images included in a plane segmentation model dataset. In at least some embodiments, the images included in the plane segmentation model dataset may be augmented with random flipping, shifting, rotation, zooming, cropping images, and/or adding noise (e.g., Gaussian noise) to images to improve the likelihood the plane segmentation model identifies which of the extracted planes includes the aortic annulus.

In at least some embodiments, one or more of the following learning techniques may be used to train the plane segmentation model: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style.

In at least some embodiments, a deep residual network or variation thereof may be used to train a model as the plane segmentation model. For example, a variation of a deep residual network referred to as ResNet may be used to train a model as the plane segmentation model. ResNet is described in, for example, “Deep Residual Learning for Image Recognition,” authored by Kaiming He, Xiangyu Zhang, Shaoqing Ren, Jian Sun, available at https://arvix.org/abs/1512.03385, the entirety of which is hereby incorporated by reference for all purposes.

Once a plane segmentation model is trained and/or once a plane segmentation model is selected, the method 400 may use the plane segmentation model to identify an implantation site (block 416). As set forth above, the aortic annulus may correspond to the implantation site for a replacement aortic valve. As such, by identifying which of the extracted planes includes the aortic annulus, the implantation site is identified.

Once the implantation site has been identified, the method 400 may include training a medical device classification model (418). The medical device classification model may be trained to determine which size medical device should be implanted into a patient.

In at least some embodiments, the medical device classification model may be trained to identify which size medical device should be implanted based on predetermined sizes of the medical device. For example, an aortic replacement valve may be manufactured in three different sizes. In these instances, the medical device classification model may be trained to determine which of the three sizes of aortic replacement valves would best fit a patient. In at least some other embodiments, the medical device classification model may be trained to determine the exact size medical device to be implanted in a patient. For example, the exact size aortic replacement valve may be output from the medical device classification model so that the exact size aortic replacement valve can be manufactured using, for example, 3-D printing techniques.

In embodiments, the medical device classification model may be trained on images included in a medical device classification model dataset. In at least some embodiments, the images included in the medical device classification model dataset may be augmented with random flipping, shifting, rotation, zooming, cropping images, and/or adding noise (e.g., Gaussian noise) to images to improve the likelihood the medical device classification model identifies an appropriately-sized medical device for a patient.

In at least some embodiments, the medical device classification model may be trained to identify one or more features of the patient's anatomy in which the medical device is to be implanted. For example, an aortic annulus may have calcification on or near the aortic annulus. In these instances, a smaller aortic implant may be required than if the aortic annulus didn't have any calcification. As such, the medical device classification model may be trained to identify what percentage of the aortic annulus includes calcification, the thickness of the calcification, and determine which size medical device should be implanted based on identified calcification. Other example features that the medical device classification model may be trained to identify include, but are not limited to, minimum radius of the aortic annulus, maximum radius of aortic annulus, area of the annulus, averages of a minimum radius and maximum radius of a plurality of aortic layers near the annulus, and/or length of the annulus centerline.

In at least some embodiments, one or more of the following learning techniques may be used to train the medical device classification model: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style.

Once a medical device classification model is trained and/or once a medical device classification model is selected, the method 400 may use the medical device classification model to identify which size medical device to implant in the implantation site (block 420). In embodiments, the medical device classification model may be similar to a visual geometry group (VGG) neural network but the medical device classification model may be in three-dimensions. The VGG neural network is described in, for example, “Very Deep Convolution Networks for Large-Scale Image Recognition,” authored by Karen Simonyan and Andrew Zisserman, and available at https://arvix.org/pdf/1409.1556.pdf, the entirety of which is hereby incorporated by reference for all purposes. In at least some embodiments, the medical device classification model may output a 3-D model structure indicating the determined size medical device that should be implanted into a patient.

In at least some embodiments, the method 400 may include extracting a pool of features from one or more of the models of method 400 that are then used to train the one or more of the models of method 400 (block 422). In embodiments where the method 400 is used to determine a size of an aortic replacement valve for a patient, exemplary features that may be extracted include, but are not limited to, minimum radius of the aortic annulus, maximum radius of aortic annulus, area of the annulus, averages of a minimum radius and maximum radius of a plurality of aortic layers near the annulus, and/or length of the annulus centerline. As an example, annulus sizing features may be extracted from the plane segmentation model; some of the annulus sizing features may be determined based on voxel intensities, which may correspond to calcification of a subject's anatomy. The extracted annulus sizing features may then be used to train the plane segmentation model. As another example, some features that don't have a particular physical meaning may be extracted from one or more layers (e.g., the last layer) of the medical device classification model, which can then be used to train the medical device classification model. These are only examples, however, and not meant to be limiting.

The illustrative method 400 shown in FIG. 4 is not intended to suggest any limitation as to the scope of use or functionality of embodiments of the present disclosure. Neither should the illustrative method 400 be interpreted as having any dependency or requirement related to any single step or combination of steps illustrated therein. Additionally, various steps depicted in FIG. 4 may be, in embodiments, integrated with various ones of the other steps depicted therein (and/or steps not illustrated), all of which are considered to be within the ambit of the present disclosure.

FIG. 5 is a flow diagram of a method 500 for training an annulus segmentation model, in accordance with embodiments of the present disclosure. As set forth above, the plane segmentation model may be trained to identify which of the extracted planes from block 412 of method 400 includes the aortic annulus.

In embodiments, the plane segmentation model may be trained on images included in a plane segmentation model dataset. In at least some embodiments, the images included in the plane segmentation model dataset may be augmented (block 502). For example, the plane segmentation model dataset may be augmented with random flipping, shifting, rotation, zooming, cropping images, and/or adding noise (e.g., Gaussian noise) to images to improve the likelihood the plane segmentation model identifies which of the extracted planes includes the aortic annulus.

As set forth above, the extracted planes may begin at a first end of the patient's anatomy relative to the centerline, extend lengthwise through the centerline, and terminate at a second end of the patient's anatomy that is on the opposite side of the centerline relative to the first end. In embodiments, each of the extracted planes may have the same thickness that extends parallel to the centerline. Further, each of the extracted planes may be divided into voxels. In embodiments, the method 500 for training the plane segmentation model may include determining an intensity value for each voxel included in an extracted plane (block 504). The method 500 may include aggregating the intensity values for each voxel included in an extracted plane (block 506). And, based on a local extrema of the aggregated value, an aortic annulus may be determined (block 508). In embodiments, the local extrema corresponding to an aortic annulus may be the extracted plane having a minimum value of the aggregated intensities, as depicted in FIG. 6A.

FIG. 6A is a computed tomography scan of an exemplary aorta 602 and FIG. 6B is a graph 604 depicting aggregated intensity values for the exemplary aorta, in accordance with embodiments of the present disclosure. As illustrated, the aorta 602 has been segmented from other portions of the patient's anatomy using, for example, the embodiments discussed above in relation to FIG. 4, blocks 406, 408. A centerline 606 has also been extracted using, for example, the embodiments discussed above in relation to FIG. 4, block 410. Furthermore, a plurality of planes 608A, 608B, 608C have been extracted using, for example, the embodiments described above in relation to FIG. 4, block 412.

As stated above, the graph depicts aggregated intensity values for the exemplary aorta 602. And, the aggregated intensity values for each of the extracted planes 608A, 608B, 608C has been determined in the graph 604. Specifically, the aggregated intensity value 610A corresponds to the aggregated intensity value for the plane 608A; the aggregated intensity value 6108 corresponds to the aggregated intensity value for the plane 608B; and, the aggregated intensity value 610C corresponds to the aggregated intensity value for the plane 608C. As shown, the aggregated intensity value 610C corresponding to the plane 608A is a local extrema (i.e., a minimum). As set forth above in relation to FIG. 4, block 416, because this is a local extrema, a plane segmentation model may determine the plane 608C to be the aortic annulus. And, based on the identification of the aortic annulus, an appropriate size medical device can be determined using, for example, a medical device classification model such as the one described above in relation to FIG. 4, block 420.

As a result of the embodiments discussed herein, a size of a medical device to be implanted in a patient may be determined more accurately in comparison to conventional applications.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A method for determining a size of a medical device to be implanted in a patient, the method comprising:
receiving a plurality of images, each of the plurality of images including a representation of a portion of a patient's anatomy in which the medical device is to be implanted;
extracting a centerline of the representation of the patient's anatomy from the plurality of images;
extracting planes orthogonal to the centerline;
identifying, using a segmentation model that segments the extracted planes, an implantation site; and
determining, using a medical device size classification model that classifies the implantation site, a size of the medical device to be implanted at the implantation site.

2. The method of claim 1, the patient's anatomy being an aorta, the area of interest being a representation of an annulus of the aorta, and the size of the medical device being one of three sizes.

3. The method of claim 1, further comprising extracting features of the patient's anatomy from the representation of a patient's anatomy using a layer of the medical device size classification model.

4. The method of claim 3, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

5. The method of claim 1, further comprising training the medical device size classification model using a classification dataset.

6. The method of claim 5, further comprising augmenting the classification dataset by randomly flipping, shifting, rotation, zooming, cropping images, and/or adding noise to images included in the classification dataset.

7. The method of claim 1, further comprising training the segmentation model using a segmentation dataset by:
determining an intensity value for each voxel in each extracted plane of the segmentation dataset;
aggregating, for each plane, the intensity values; and
identifying local extrema among the aggregated intensity values.

8. The method of claim 7, wherein identifying local extrema comprises identifying a local minimum, the local minimum corresponding to an annulus.

9. The method of claim 7, further comprising augmenting the segmentation dataset by randomly flipping, shifting, rotation, zooming, cropping images, and/or adding noise to images included in the segmentation dataset.

10. The method of claim 1, further comprising preprocessing the plurality of images, the preprocessing including at least one of: scaling the plurality of images so the plurality of images are uniform and removing at least some portions of the plurality of images that do not include the representation of the patient's anatomy.

11. A non-transitory computer readable medium having a computer program stored thereon for determining which size medical device to implant in a patient, the computer program comprising instructions for causing one or more processors to:
receive a plurality of images, each of the plurality of images including a representation of a portion of a patient's anatomy in which the medical device is to be implanted;
extract a centerline of the representation of the patient's anatomy from the plurality of images;
extract planes orthogonal to the centerline;
identify, using a segmentation model that segments the extracted planes, and implantation site; and
determine, using a medical device size classification model that classifies the implantation site, a size of the medical device to be implanted at the implantation site.

12. The non-transitory computer readable medium of claim 11, the patient's anatomy being an aorta, the area of interest being a representation of an annulus of the aorta, and the size of the medical device being one of three sizes.

13. The non-transitory computer readable medium of claim 11, the computer program comprising instructions for causing the one or more processors to extract features of the patient's anatomy from the representation of a patient's anatomy using a layer of the medical device size classification model.

14. The non-transitory computer readable medium of claim 13, the patient's anatomy being an aorta and the extracted features including at least one of: a minimum radius of an annulus of the aorta, a maximum radius of the annulus, an area of the annulus, averages of a minimum radius and maximum radius of a plurality of layers near the annulus, a percentage of the annulus including calcium, and a length of the annulus centerline.

15. The non-transitory computer readable medium of claim 11, the computer program comprising instructions for causing the one or more processors to train the medical device size classification model using a classification dataset.

16. The non-transitory computer readable medium of claim 15, the computer program comprising instructions for causing the one or more processors to augment the classification dataset by randomly flipping, shifting, rotation, zooming, cropping images, and/or adding noise to images included in the classification dataset.

17. The non-transitory computer readable medium of claim 11, the computer program comprising instructions for causing the one or more processors to train the segmentation model by:
determine an intensity value for each voxel in each extracted plane;
aggregate, for each plane, the intensity values; and
identify local extrema among the aggregated intensity values.

18. The non-transitory computer readable medium of claim 17, wherein to identify local extrema, the computer identifies a local minimum, the local minimum corresponding to an annulus.

19. The non-transitory computer readable medium of claim 17, the computer program comprising instructions for causing the one or more processors to augment the segmentation dataset by randomly flipping, shifting, rotation, zooming, cropping images, and/or adding noise to images included in the segmentation dataset.

20. The non-transitory computer readable medium of claim 11, the computer program comprising instructions for causing the one or more processors to preprocess the plurality of images, the preprocessing including at least one of: scaling the plurality of images so the plurality of images are uniform and removing at least some portions of the plurality of images that do not include the representation of the patient's anatomy.
